(19) United States
(12) Patent Application Publication
(10) Pub. No.: US 2025/0041551 A1
(43) Pub. Date: Feb. 6, 2025

(54) CONTINUOUS POSITIVE AIRWAY PRESSURE DEVICES AND METHODS OF USE

(71) Applicant: Therapeutic Innovations Inc., Sutton, MA (US)

(72) Inventors: Dirk R. Albrecht, Chatham, NJ (US); Solomon A. Mensah, Sutton, MA (US)

(21) Appl. No.: 18/811,190

(22) Filed: Aug. 21, 2024

Related U.S. Application Data
(63) Continuation-in-part of application No. PCT/US2024/016823, filed on Feb. 22, 2024.
(60) Provisional application No. 63/488,968, filed on Mar. 7, 2023.
(60) Provisional application No. 63/488,745, filed on Mar. 6, 2023.
(60) Provisional application No. 63/486,461, filed on Feb. 22, 2023.

(51) Int. Cl. A61M 16/10; A61M 16/00; A61M 16/16

(52) U.S. Cl. CPC A61M 16/1095; A61M 16/0063; A61M 16/109; A61M 16/16; A61M 2205/3666; A61M 2205/50; A61M 2205/8206; A61M 2210/0618

(57) ABSTRACT

Disclosed herein is a CPAP device. In some embodiments, the CPAP device may be a bCPAP device. In some embodiments, the device may have one or more features that may assist in heating efficiency. In some embodiments, an air mixture is sent to a humidifier via an inlet conduit prior to introduction to a patient. In some embodiments, a water chamber of the humidifier may be recessed within a compartment. In some embodiments, waste heat from one or more components of the device may be used to pre-heat the air mixture in the inlet conduit.

### CROSS-REFERENCE TO RELATED APPLICATIONS

This application is a continuation-in-part of International Application No. PCT/US2024/016823, filed Feb. 22, 2024, which claims the benefit of U.S. Provisional Application No. 63/488,968, filed Mar. 7, 2023, U.S. Provisional Application No. 63/488,745, filed Mar. 6, 2023, and U.S. Provisional Application No. 63/486,461, filed Feb. 22, 2023. The contents of these applications are incorporated herein by reference in their entirety.

### FIELD

Disclosed embodiments are related to continuous positive airway pressure (CPAP) devices and associated methods of use.

### BACKGROUND

Continuous positive airway pressure (CPAP) devices are used to treat respiratory disorders. These devices may deliver air and help keep the lungs partially inflated. In this manner, the CPAP device may reduce or, in some instances, help prevent the collapse of the patient's upper airways, thus making it easier for the patient to breathe.

### SUMMARY

According to some aspects, a continuous positive airway pressure (CPAP) device is provided. In some embodiments, the CPAP device may include a humidifier comprising a water chamber, a heater configured to heat the water chamber, an inlet conduit configured to direct an air mixture into the water chamber, and a compressor configured to move the air mixture through the inlet conduit. In some embodiments, the compressor and the inlet conduit are positioned relative to one another such that waste heat from the compressor is configured to pre-heat the air mixture in the inlet conduit.

According to some aspects, a continuous positive airway pressure (CPAP) device is provided. In some embodiments, the CPAP device may include a housing having an internal volume. In some embodiments, at least a portion of the compressor is disposed in the internal volume of the housing. In some embodiments, the CPAP device may include a humidifier that includes a water chamber. In some embodiments, the CPAP device may include a compartment configured to receive at least a portion of the water chamber. In some embodiments, the compartment may be positioned external to the internal volume of the housing. In some embodiments, the CPAP device may include a heater configured to heat the water chamber, where at least a portion of the heater is disposed within the compartment. In some embodiments, the CPAP device may include a compressor configured to move an air mixture to the water chamber. In some embodiments, the CPAP device may include an inspiratory tube configured to lead the air mixture from the water chamber to a patient respiratory interface.

According to some aspects, a method for operating a continuous positive airway pressure (CPAP) device is provided. In some embodiments, the method includes directing, with a compressor, an air mixture through an inlet conduit to a water chamber. In some embodiments, the method includes pre-heating the air mixture in the inlet conduit with waste heat from the compressor. In some embodiments, the method includes humidifying and heating the air mixture in the water chamber. In some embodiments, the method includes directing the humidified and heated air mixture through an inspiratory tube to a patient respiratory interface.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF DRAWINGS

FIG. 1 illustrates a CPAP device according to one embodiment, where the housing is shown in phantom to reveal internal components;

FIG. 2 illustrates a top view of the CPAP device according to one embodiment, where a top portion of the housing is hidden from view;

FIG. 3 depicts the CPAP device according to one embodiment; and

FIG. 4 illustrates a cross-section of the CPAP device according to one embodiment.

### DETAILED DESCRIPTION

Continuous positive airway pressure (CPAP) devices may be used to treat various respiratory disorders, including neonatal respiratory disorders. For example, CPAPs can be used for the treatment of infant Respiratory Distress Syndrome (iRDS). Annually, the disorder accounts for approximately 50 percent of infant deaths worldwide. A majority of these deaths occur in developing countries.

The inventor(s) have recognized that many of these deaths may occur due to a lack of medical technology in these countries due to a significant cost barrier. Many conventional CPAP devices do not meet the price point that would make them accessible worldwide. The inventor(s) have appreciated that a CPAP device that is more cost effective to manufacture and operate could greatly reduce the number of infant deaths in developing countries.

The inventor(s) have recognized several limitations in conventional CPAP devices that make these devices difficult to repair and/or maintain over time, energy inefficient, not portable, expensive, and/or difficult to use. Together, these limitations have limited the deployment of CPAP devices in more resource-scarce environments (e.g., developing countries).

For example, the inventor(s) have appreciated that conventional CPAP devices that include an integrated heating device to deliver warm air to patients typically experience appreciable heat losses throughout the device. To compensate for these heat losses, conventional heating devices are often run at higher power settings, thus consuming more electricity and reducing energy efficiency. As a result, many conventional CPAP devices often require an electrical connection to an external power source (e.g., building mains) to receive sufficient electrical power. In other words, conventional CPAP devices are seldom configured to run on batteries for extended periods of time (e.g., more than 3 hours), particularly if a heating device is included.

In view of the foregoing limitations of conventional CPAP devices, the inventors have recognized the need for a CPAP machine which is more energy efficient to operate when compared to traditional CPAP machines.

According to some aspects described herein, a CPAP machine may include one or more features that reduce the energy needed to operate. The components of the CPAP machine and their functionality, especially in relation to improving energy efficiency, will be discussed below.

In some embodiments, the CPAP device may be configured to deliver a desired amount of air flow at 37° C. with a relative humidity of 100% to a patient. In some embodiments, the CPAP machine is configured to compress, heat and humidify air and deliver it to a patient. In some embodiments, the CPAP machine may be operated with an internal battery.

In some embodiments, the CPAP device may be configured to operate as a bubble CPAP device. For example, the CPAP device may include a pressure bottle to contain a column of water and into which an expiratory end of tubing connected to the patient is submerged to regulate the pressure of air flowing through the CPAP device and to the patient. The air supplied by the CPAP device may have a composition substantially similar to or the same as the air in the ambient environment, or may be mixed with oxygen from a separate oxygen source using an air/oxygen mixer.

In some embodiments, the CPAP device may include a heating device configured to interact with a bubbling humidifier. For example, the heating device may include a base plate (which may be metal) with at least one heater and a water chamber with an inlet and an outlet. During operation, the water chamber may contain water that is heated by the base plate via the heater(s). Air supplied, for example, from an air pump or compressor, may be directed into the water chamber and may be heated/humidified within the water chamber before being vented through the outlet and to the patient.

In some embodiments, the water chamber may be disposed in a cavity, also referred to as a compartment, of the housing. According to one aspect, positioning the water chamber within a compartment may help to decrease heat loss of the water chamber, which in turn may reduce the electrical power for the heating device to heat the air to a desired temperature setpoint. In some embodiments, thermal insulation may be disposed around the water chamber to help reduce heat losses. In some embodiments, an air gap and/or insulating materials may serve as thermal insulation. The compartment may have an open top to allow the water chamber to be placed into the compartment and removed from the compartment. In some embodiments, the compartment is located external to the internal volume of the housing. In other words, in some embodiments, the compartment does not share a volume with an internal volume of the housing. Other components of the CPAP device such as a battery and/or a compressor may be located within the internal volume of the housing.

The water chamber may be removably coupled to the base plate via a coupling mechanism (e.g., via a tool-less mechanism, such as a snap-fit or twist-and-lock mechanism). The coupling mechanism may securely couple the water chamber to the base plate such that the water chamber remains coupled to the base plate when the CPAP device is subjected to vibration and/or other external forces (e.g., during shipping, and transportation). In some embodiments, the water chamber may hold up to 450 mL of water. It should be appreciated that the water chamber may have any suitable volume.

In some embodiments, the humidifier may have a metal base. From the metal base, the water inside the water chamber may be heated. As the metal base heats the water, the hot liquid may rise up, while the cooler liquid may sink to the bottom to be heated by the metal base. When the hot water rises to the top, the hot water may transfer heat to the air. The tubes that deliver air to the patient may be positioned at the top of the water chamber, so as the air is heated, the hot air rises and enters the tubing device.

According to one aspect, in some embodiments, waste heat from one or more components of the CPAP device may be used to pre-heat an air mixture prior to introducing to a patient. Such an arrangement may help to increase heating efficiency. In some embodiments, one or more components that produce waste heat may be positioned close enough to an inlet conduit to heat an air mixture within the inlet conduit. The inlet conduit may, in some embodiments, lead to an air mixture to a humidifier prior to being introduced to a patient.

In some embodiments, the CPAP device may be powered by one or more batteries for an extended period of time. It should be appreciated that the CPAP device may also receive electrical power via an electrical connection to an external power source (e.g., a wall outlet provided AC power and connected to the mains of a building). In some embodiments, when the CPAP device is operated with an external power source, the internal battery may be charged during operation.

The internal battery may include a current-limiting resistor. In some embodiments, the current-limiting resistor may be a ceramic heater cartridge. The current-limiting resistor may protect the internal battery from being damaged by excess current. The current-limiting resistor may both dampen the current and provide waste heat. In some embodiments, the current-limiting resistor may be positioned within a heater and the waste heat may be used the heat the water chamber of the humidifier. In some embodiments, the current-limiting resistor may be positioned within a housing. The current-limiting resistor may assist in pre-heating an air mixture within the inlet conduit.

In some embodiments, the heater may comprise a solid-state heater. The heater may be a 12V ceramic heater (e.g., 10-40 W) that operates using a digital signal input (0V/5V TTL). The heater may use an on/off at a pulse width modulation (PWM) frequency of <=0.2 Hz. In some implementations, a control circuit (e.g., one or more MOSFETs/transistors) may be assembled, in part, onto a protoboard (or a printed circuit board—also referred to as a PCB) to turn the heater on/off using a digital signal. Appropriate wiring may be used to electrically connect the protoboard to the heater. In some implementations, wiring may be soldered directly to the protoboard and include shrink tube insulation. In some implementations, the wiring may include one or more connectors (e.g., JST-XH connectors) to more easily connect and/or disconnect the heater from the protoboard.

In some embodiments, the CPAP device may include a processor and one or more temperature sensors, which operate in combination with the heating device to regulate the temperature of the air delivered to the patient (e.g., the processor, the heater(s), and the temperature sensor(s) are communicatively coupled to each other). In some implementations, the processor may be configured to execute a proportional-integral-derivative (PID) control loop algorithm that uses temperature data acquired by the temperature sensor(s) as feedback to adjust the power of the heating device to maintain a desired air temperature according to a predetermined temperature setpoint (e.g., a user selected setpoint of 37° C.). In some implementations, a heat transfer model may be incorporated into the algorithm, in part, to determine an initial setting for a heater power that compensates for heat losses in the CPAP device and/or to determine an update to the heater power during each loop of the PID control loop. Of course, although the user may select 37° C. as the desired temperature, any temperature may be selected.

According to some aspects, the CPAP machine is configured to increase heating efficiency by reducing the convective heat loss through the inspiratory tubing or housing. For example, in some embodiments, the inspiratory tubing is thermally insulated. In some embodiments, the housing may insulate the components, and the heat they produce, within the housing. This may differ from conventional CPAP devices, which may not be designed with an emphasis on heating efficiency. In some embodiments, the inventors have recognized that retaining heat within the housing, and transferring the heat to an inlet air supply, may increase the efficiency of the device.

In some embodiments, the CPAP device may include a mounting mechanism to removably couple a water chamber to the heater. As described above, the water chamber may function as a humidifier to both warm and humidify the air provided to the patient. The heater may include, for example, an aluminum disk with heaters securely fixed to the disk.

The heater may be configured to accommodate the selected heaters used for the CPAP device. For example, the heaters may be 6×20 mm 3D printer heaters for their availability and low cost. The mounting mechanism may provide sufficient mechanical strength to retain the water chamber to the base plate when the CPAP device is subjected to vibration (e.g., during shipping, transportation). Thermal insulation may also be disposed around the base plate to reduce heat losses to the surroundings and, hence, increase heat transfer to the water stored in the water chamber. The thermal insulation will be further discussed below.

Turning to the figures, specific non-limiting embodiments are described in further detail. It should be understood that the various devices, components, features, and methods described relative to these embodiments may be used either individually and/or in any desired combination as the disclosure is not limited to only the specific embodiments described herein.

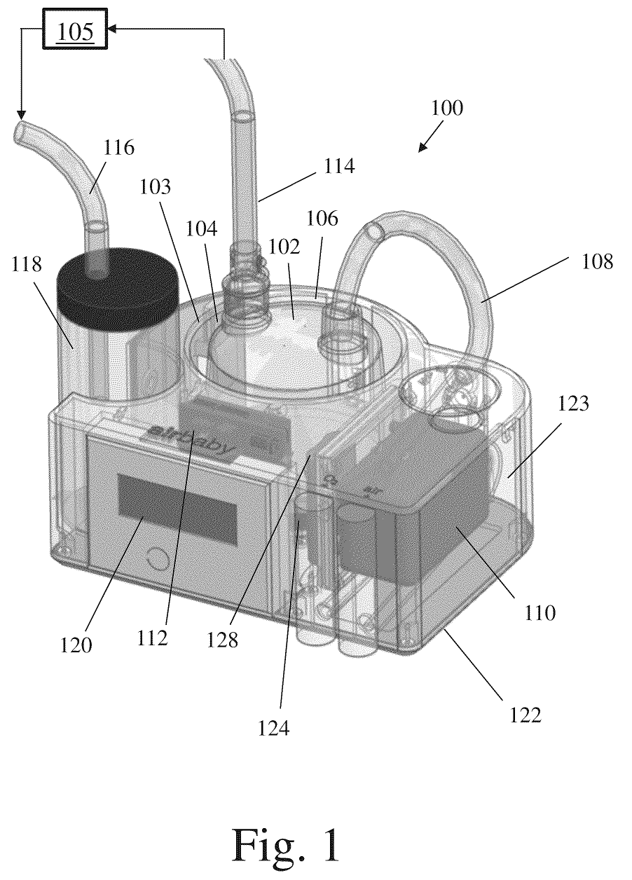

FIG. 1 illustrates a CPAP device with and its internal components according to one embodiment. In this illustration, the housing 122 is depicted as clear such that the internal components positioned within an internal volume 123 of the housing are visible. In some embodiments, the housing may be clear, opaque, or any combination thereof. A compressor 110, a battery 128, a printed circuit board (PCB) 112, and a portion of the inlet conduit 108 may be positioned within the internal volume 123 of the housing 122. In some embodiments, the water chamber 102 may be disposed within an indent 103 in the housing, also referred to herein as a compartment.

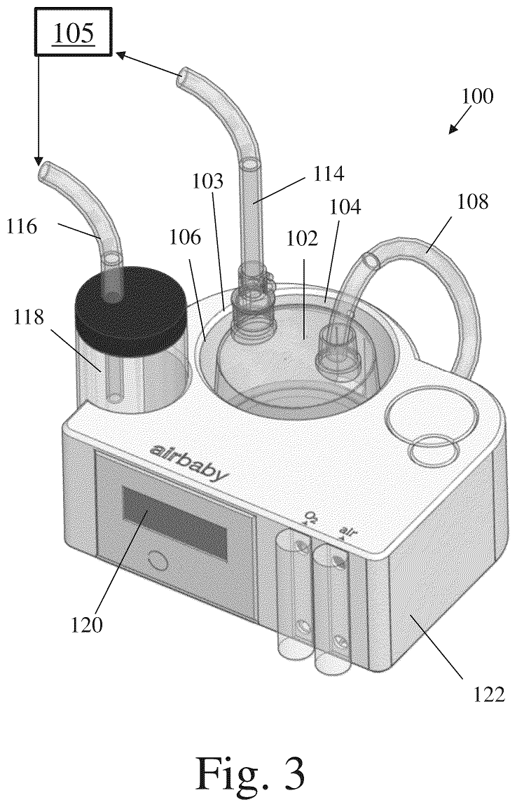

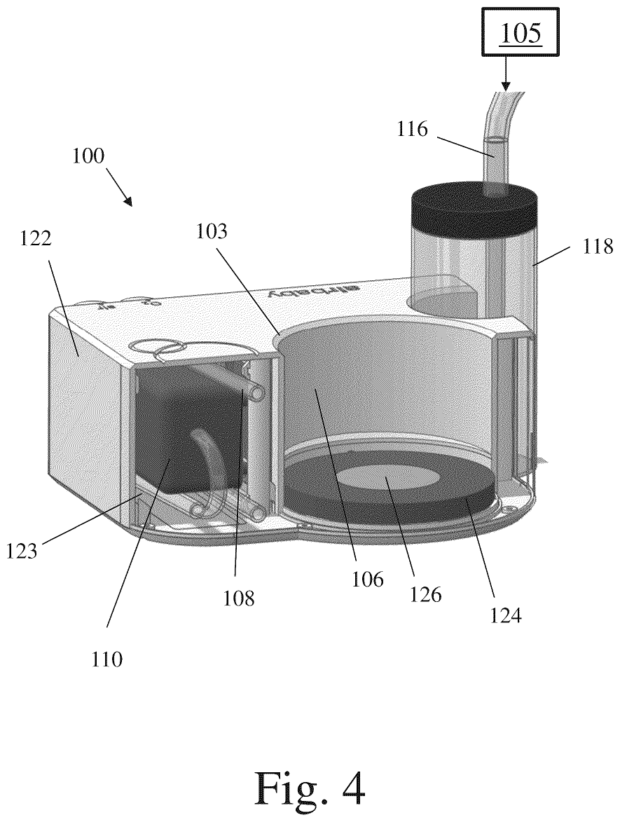

As seen in FIG. 3, the compartment 103 may have an open top. As seen in FIGS. 3 and 4, in some embodiments, the compartment 103 may be positioned external to an internal volume 123 of the housing. In other words, in some embodiments, the compartment 103 does not share a volume with the internal volume of the housing. The CPAP device may include an inspiratory tube 114 which leads from the water chamber 102 to a patient respiratory interface 105. In some embodiments, the patient respiratory interface is a nasal interface. The patient respiratory interface may then lead to an expiratory tube 116 which may terminate in a pressure accumulator 118.

In some embodiments, a display screen 120 may be disposed on the outside of the housing 122. The display screen may display operation information including, but not limited to, air temperature, air humidity, water temperature, and battery level.

In some embodiments, the pressure accumulator 118 may be disposed outside of the housing 122. The CPAP device may be a bubble CPAP where the expiratory tube is submerged in water within the pressure accumulator. The volume of water present within the pressure accumulator may control the pressure of the air mixture delivered to the patient respiratory interface.

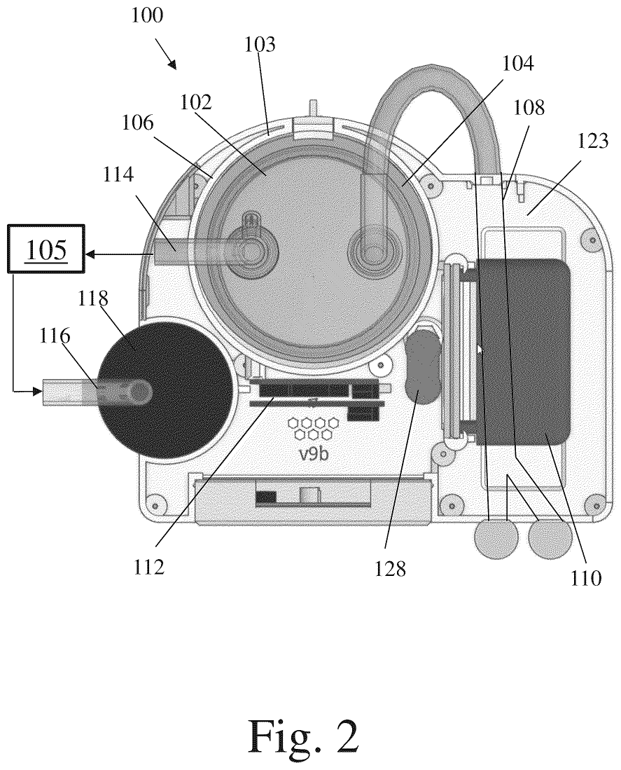

FIG. 2 illustrates a top view of the CPAP device according to one embodiment. The top surface of the housing has been hidden from view in order to visualize the internal components of the device. In this figure, the relative placement of the inlet conduit 108, compressor 110, PCB 112, and battery 128 may be seen. In some embodiments, the heater may account for a large portion of the energy usage in the device. The inventors have thus recognized that reducing the burden of the heater would increase the efficiency of the device. The inventors have recognized that components such as the compressor 110, PCB 112, and battery 128 may release waste heat during operation. Accordingly, in some embodiments, the inlet conduit 108 is positioned such that the air mixture flowing through the inlet conduit 108 may be pre-heated by waste heat from one or more components of the CPAP device. In some embodiments, the portion of the inlet conduit 108 which is disposed within the housing 122 may be designed to transfer energy from the air in the housing 122 to the air mixture within the inlet conduit 108. In some embodiments, at least the portion of the inlet conduit 108 which is disposed in the housing 122 may have a thin wall to improve heat transfer. In some embodiments, at least the portion of the inlet conduit 108 which is disposed in the housing 122 may be constructed from a conductive material.

The inventors have realized that positioning one or more waste heat generating components (e.g. compressor 110, PCB 112, and/or battery 128) in the same confined volume as the inlet conduit 108 may allow the waste heat to pre-heat the contents of the inlet conduit 108. As mentioned above, in some embodiments, the housing may be configured to encapsulate compressor 110, PCB 112, battery 128, inlet conduit 108, or any combination of these components. Therefore, the waste heat may increase the temperature within the housing and this heat may be transferred to the contents of the inlet conduit 108. Additionally, the waste heat generating components may be positioned in close proximity to the inlet conduit 108. As seen in FIG. 2, the inlet conduit 108 is positioned adjacent to the compressor 110. The close proximity of the compressor 110 to the inlet conduit 108 may assist the waste heat produced by the compressor 110 in pre-heating the contents of the inlet conduit 108. Although the compressor 110 is disposed adjacent to the inlet conduit 108 in this embodiment, other embodiments are contemplated in which, alternatively or in addition, the PCB 112 and/or the battery 128 are disposed adjacent to the inlet conduit 108.

The inventors have contemplated that the addition of any relevant waste heat producing component may be positioned adjacent to, or within the same volume as, any portion of the inlet conduit 108 may increase the efficiency of the device. This may include filters, pumps, sanitizing steps, energy manipulators, or any other component which may be used in a CPAP device.

FIG. 3 depicts the CPAP device according to one embodiment. In some embodiments, the CPAP device may include a heating device configured to operate as a bubbling humidifier. For example, the heating device may include a base plate (which may be metal) with at least one heater and a water chamber with an inlet and an outlet. During operation, the water chamber may contain a column of water that is heated by the base plate via the heater(s). Air supplied, for example, from an air pump or compressor, may be directed into the column of water and heated/humidified directly within the water before being vented through the outlet and to the patient. The water chamber may be disposed in a compartment recessed within the housing. In some embodiments, the compartment does not share an internal volume with the volume of the housing. Thermal insulation may be disposed around the water chamber to reduce heat losses, which, in turn, reduces the electrical power for the heaters to heat the air to a desired temperature setpoint. As mentioned above, the water chamber may be removably coupled to the base plate via a coupling mechanism (e.g., via a tool-less mechanism, such as a snap-fit or twist-and-lock mechanism). The coupling mechanism may securely couple the water chamber to the base plate such that the water chamber remains coupled to the base plate when the CPAP device is subjected to vibration and/or other external forces (e.g., during shipping, transportation).

As mentioned above, in some embodiments, the CPAP device may have a water chamber disposed on a hot plate which is configured to heat the water. When pressurized air passes through the water chamber, the air is heated and humidified. Accordingly, in some embodiments, the water chamber may be considered a humidifier. The inventors have recognized that heat loss to the environment may increase the energy required to operate the device. The hot plate may use a large amount of energy.

According to some aspects, the CPAP device may be configured to decrease heat lost from the humidifier chamber. In some embodiments, the water chamber 102 is at least partially recessed within a compartment 103. In some embodiments, the compartment may be sized such that, with the water chamber 102 of the humidifier positioned within the compartment 103, an air gap 104 may be present between an external surface of the water chamber 102 and the wall 106 of the compartment. The air gap may serve as an insulator to help decrease heat loss from the water chamber 102. In some cases, the air gap may allow a larger portion of the water chamber to be viewable by an operator, e.g. to monitor water levels.

In some embodiments, an insulation material such as rubber may be included within the compartment, e.g. positioned on the wall 106 of the compartment. It should be understood that any insulating material may be used to assist in retaining heat within the water chamber 102.

In some embodiments, the wall 106 of the compartment fully surrounds the water chamber, e.g. completely surrounding a sidewall of the water chamber. In some embodiments, the wall 106 of the compartment may be relatively deep such that a majority of the water chamber is surrounded by the compartment wall 106. In some embodiments, the compartment wall 106 extends at least halfway up a height of the water chamber 102 when the water chamber is positioned in the compartment. In some embodiments, the compartment wall 106 extends at least the height of the water chamber 102 such that the water chamber 102 is completely recessed within the compartment.

FIG. 4 illustrates a cross-section of the CPAP device. In this figure, a heater 126 in the form of a heater plate is disposed within the compartment. The heater 126 may be surrounded by an insulator 124. This insulator 124 may differ from the air gap 104 disposed between the water chamber 102 and the compartment wall 106. In some embodiments, the insulator 124 may be ring shaped. The insulator may cover all sides of the heater 126 except the side which contacts the water chamber 102. In some embodiments, the insulator 124 may cover all sides of the heater 126 except the top side, as is depicted in FIG. 4. The insulator 124 may be configured to direct the heat produced by the heater 126 into the water chamber 102. The insulator may help to decrease heat loss from the heater. In some embodiments, the insulator 124 may be disposed below the heater 126 which may comprise a base plate. In some embodiments, the insulator 124 may at least partially circumscribe the heater 126. In some embodiments, an upper surface of the heater 126 and the insulator 124 may be substantially flush, as depicted in FIG. 4.

In some embodiments, the water chamber of the humidifier may have a metal base which may be formed of thin aluminum. The metal base may be shaped to bow downwards in the center, i.e., it is not flat. In this way, pressure forcing the water chamber 102 downward may flatten the metal base, thus making good physical and thermal contact with the heater 126.

As discussed above, one or more features of the CPAP device may allow the device to require significantly lower power use than conventional CPAP devices. In some embodiments, the total power use of the CPAP device during start up may be less than or equal to 40 W, 45 W, 50 W, 55 W, 60 W, 65 W, 70 W, 75 W, 80 W, 85 W, 90 W, 95 W, or 100 W. In some embodiments, the total power use of the CPAP device during start up may be 20 to 60 W, 25 to 55 W, 30 to 50 W, or 35 to 45 W.

It should be appreciated that, in some embodiments, the total power use of the CPAP device may be higher during start up than at a steady state run. In some embodiments, the total power use of the CPAP device at a steady run state may be less than or equal to 10 W, 15 W, 20 W, 25 W, 30 W, 35 W, 40 W, 45 W, 50 W, 55 W, 60 W, 65 W, 70 W, 80 W, 90 W, or 100 W. In some embodiments, the total power use of the CPAP device at a steady run state may be 5 to 35, 10 to 30, 15 to 25, or 17 to 24 W.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A continuous positive airway pressure (CPAP) device comprising:
a humidifier comprising a water chamber;
a heater configured to heat the water chamber;
an inlet conduit configured to direct an air mixture into the water chamber; and
a compressor configured to move the air mixture through the inlet conduit,
wherein the compressor and the inlet conduit are positioned relative to one another such that waste heat from the compressor is configured to pre-heat the air mixture in the inlet conduit.

2. The device of claim 1, further comprising a housing, wherein at least a portion of the inlet conduit is disposed in an internal volume of the housing.

3. The device of claim 2, wherein the compressor is disposed in the internal volume of the housing.

4. The device of claim 2, wherein a printed circuit board (PCB) is disposed in the internal volume of the housing.

5. The device of claim 1, wherein the inlet conduit is adjacent to the compressor.

6. The device of claim 1, further comprising a battery, wherein the battery is configured to power the CPAP device.

7. The device of claim 1, further comprising:
a pressure accumulator; and
an inspiratory tube configured to lead the air mixture from the water chamber to a patient respiratory interface, and further comprising an expiratory tube configured to lead the air mixture from the patient respiratory interface to the pressure accumulator.

8. The device of claim 7, wherein the patient respiratory interface comprises a nasal interface.

9. A continuous positive airway pressure (CPAP) device comprising:
a housing having an internal volume;
a humidifier comprising a water chamber;
a compartment configured to receive at least a portion of the water chamber, wherein the compartment is positioned external to the internal volume of the housing;
a heater configured to heat the water chamber, wherein at least a portion of the heater is disposed within the compartment;
a compressor configured to move an air mixture to the water chamber, wherein at least a portion of the compressor is disposed in the internal volume of the housing; and
an inspiratory tube configured to lead the air mixture from the water chamber to a patient respiratory interface.

10. The device of claim 9, wherein with the water chamber positioned in the compartment, an exterior surface of the water chamber is spaced from a wall of the compartment to define an insulating air gap disposed between the water chamber and the wall of the compartment.

11. The device of claim 10, wherein with the water chamber positioned in the compartment, the wall extends at least halfway up a height of the water chamber.

12. The device of claim 9, further comprising an insulator disposed beneath the heater.

13. The device of claim 9, further comprising an inlet conduit configured to direct an air mixture into the water chamber, wherein at least a portion of the inlet conduit is disposed in the internal volume of the housing.

14. The device of claim 9, further comprising a battery, wherein the battery is configured to power the CPAP device.

15. The device of claim 9, further comprising an expiratory tube configured to lead the air mixture from the patient respiratory interface to a pressure accumulator.

16. A method for operating a continuous positive airway pressure (CPAP) device comprising:
directing, with a compressor, an air mixture through an inlet conduit to a water chamber;
pre-heating the air mixture in the inlet conduit with waste heat from the compressor;
humidifying and heating the air mixture in the water chamber; and
directing the humidified and heated air mixture through an inspiratory tube to a patient respiratory interface.

17. The method of claim 16, further comprising pre-heating the air mixture in the inlet conduit with waste heat from a printed circuit board (PCB).

18. The method of claim 16, further comprising heating the water chamber with a heater.

19. The method of claim 16, further comprising:
pre-heating the air mixture in the inlet conduit with waste heat from an internal battery of the CPAP device.

20. The method of claim 16, further comprising:
charging an internal battery while operating the CPAP device using an external power source, wherein charging the internal battery generates waste heat; and
pre-heating the air mixture in the inlet conduit with waste heat from the internal battery.
